(19) United States
(12) Patent Application Publication
(10) Pub. No.: US 2015/0343237 A1
(43) Pub. Date: Dec. 3, 2015

(54) METHOD AND POSITION DETERMINATION SYSTEM FOR DETERMINING A POSITION OF A TARGET REGION OF A PATIENT TO BE IRRADIATED IN AN IRRADIATION DEVICE

(71) Applicant: Siemens Aktiengesellschaft, Muenchen (DE)

(72) Inventors: Annemarie Hausotte, Erlangen (DE); Martin Requardt, Nuernberg (DE)

(73) Assignee: SIEMENS AKTIENGESELLSCHAFT, Muenchen (DE)

(21) Appl. No.: 14/729,523

(22) Filed: Jun. 3, 2015

(30) Foreign Application Priority Data
Jun. 3, 2014 (DE) 102014210458.0

(51) Int. Cl. A61N 5/10; A61B 6/04; A61B 6/12; A61B 5/055; A61B 19/00

(52) U.S. Cl. CPC A61N 5/1039; A61B 5/055; A61B 19/54; A61B 6/12; A61N 5/1049; A61B 6/0407; A61B 2019/5454; A61B 2019/5466; A61N 2005/1061

(57) ABSTRACT

In a method for determining a position of a target region of a patient to be irradiated in an irradiation device, a patient is positioned on a patient positioning device having markers that are visible in magnetic resonance images. A magnetic resonance image data record is acquired showing a target area of the patient containing the target region, together with the markers. The magnetic resonance image data record is evaluated to determine location information describing the three-dimensional position of the target region relative to the markers. The patient positioning device with the unmoved patient is transported to the irradiation device. Target information describing the position of the target region in the irradiation device is determined from marker information indicating the position of the markers in the irradiation device, and the location information.

### BACKGROUND OF THE INVENTION

1. Field of the Invention

The invention relates to a method for determining a position of a target region of a patient to be irradiated in an irradiation device and a position determination system for implementing such a method.

2. Description of the Prior Art

Irradiation devices for radiotherapy are widely known in the prior art and are used to treat tumors and the like. During this treatment, particles, for example electrons, are accelerated by an accelerator, in particular a linear accelerator (LINAC), and then the therapeutic beam is focused on a target region of a patient, wherein, for example, the radiation source can be rotated about the patient so that the highest radiation density is actually established in the target region. For radiotherapy treatment with an irradiation device, it is essential that the correct target region is irradiated and therefore that the patient is positioned correctly for the irradiation.

For this purpose, increased use is being made of image-based positioning methods with radiotherapy treatments, to which end, for example, the irradiation device itself can comprise an image recording device, for example an X-ray image recording device. For example, an X-ray tube and an X-ray detector permitting the recording of X-ray images under different projection directions can be provided on the gantry with which the radiation source can be swiveled about the patient, in particular at a 90° angle to the radiation direction of the radiation source. In this case, two-dimensional X-ray images used to position the patient are often referred to as “portal images”, but, due to the rotatability of the recording arrangement, it is also possible to use X-ray images with different projection directions to determine a three-dimensional, computed-tomography-like image data record. X-ray image recording devices on irradiation devices generally use cone-beam geometry.

Portal images or three-dimensional X-ray image data records recorded with the image recording device of the irradiation device particularly clearly visualize the patient's bones. Such imaging of the bones is also calculated from computed-tomography planning image data records used for radiation planning so that it is possible to ensure from a comparison that the current measured image of the bones of the patient corresponds to the constellation of the bones at the time of the planning. However, one problem with radiation planning based on X-ray imaging is that it has poorer soft-tissue contrast and depicts the tissue in the target region that is actually to be irradiated poorly or not all. However, it is known that, even with the same positioning of the bones, slight displacements of the target region can occur, for example due to digestive activities and/or a full bladder and the like.

It has therefore been suggested that magnetic resonance imaging be used for the planning and/or preparation of an irradiation session, i.e. that magnetic resonance image data records be used to identify the target region to be irradiated. Magnetic resonance image data records provide an extremely good soft-tissue contrast but bones are much more difficult to depict so that the amount of work to ensure the same position with irradiation and purely magnetic-resonance-based radiation planning is disproportionately greater than when this is to be performed on a bone basis.

It has therefore been suggested that radiation planning be performed on a computed-tomography-planning image data record on which at least one magnetic resonance planning image data record has been recorded in order to compensate for the deficits of computed tomography with respect to soft-tissue contrast. It is in particular known to calculate a so-called DRR (digitally reconstructed radiograph) of the bones from the computed-tomography-planning image data record, wherein the DRR was then compared with the localization image data record recorded by the image recording device of the irradiation device, that is the portal image or the computed-tomography-like three-dimensional image data record. The drawback of this is that in principle a CT-planning image data record always must be present and the comparison with the localization image data record is ultimately performed by means of a surrogate, namely the bones. If, for example, it is wished to irradiate the prostate as a target region, reference is made to the pelvic bones which are depicted as a bony structure in the localization image data record and in the DRR. However, this could be associated with the aforementioned problems that the positional relationship of the bones to the target region is not necessarily totally constant.

It has been suggested in an alternative solution that gold markers be introduced into the target region, for example the prostate and/or a soft-tissue tumor, which are visible in both the magnetic resonance planning image data record and the localization image data record on the irradiation device and in this way also enable a comparison. This avoids the described use of the bones as a surrogate, but requires the insertion of the image markers, an intervention that could be considered to be critical or unacceptable.

### SUMMARY OF THE INVENTION

An object of the invention is to use magnetic resonance imaging to enable improved positioning of a target region of a patient to be irradiated in an irradiation device.

This object is achieved in accordance with the invention by a method for determining a position of a target region of a patient to be irradiated in an irradiation device that includes the following steps:

positioning a patient on a patient positioning device comprising markers that are visible at least in magnetic resonance imaging,

recording a magnetic resonance image data record showing a target area of the patient containing the target region to be irradiated together with the markers with a magnetic resonance device,

evaluating, preferably automatically the magnetic resonance image data record, determining location information describing the three-dimensional position of the target region relative to the markers,

transporting the patient positioning device with an unmoved patient to the irradiation device,

determining, preferably automatically, target information describing the position of the target region in the irradiation device using marker information indicating the position of the markers in the irradiation device and the location information.

Therefore, the image-based verification of the correct positioning of the patient in the irradiation device is performed not on the basis of bony structures but is focused on the soft-tissue structures of interest that are visible in the magnetic resonance image data record. This enables the complicated calculation of a bone DRR to be dispensed with. Instead, the present invention uses two-stage positioning on the target region. The patient can be supported on a magnetic-resonance-compatible patient positioning device that has markers placed on it that are visible in a magnetic resonance image. Prior to the performance of the irradiation, the patient, together with the patient positioning device, is then scanned in a magnetic resonance scanner so that a magnetic resonance image data record is acquired. This enables, in a way that is in principle known per se, the determination of the precise location of the target region to be irradiated, for example a soft-tissue tumor. However, since the markers are also visible in the magnetic resonance image data record, it is possible, in particular by automatic evaluation of the magnetic resonance image data record, to determine the three-dimensional position of the target region relative to the markers, that is the location information. The automatic evaluation can be performed using, for example, segmentation algorithms and the like.

The patient is then, without being moved relative to the patient positioning device, in particular relative to the markers, transported to the irradiation device where known or and/or determinable marker information is used. The marker information describes the position of the markers in the radiation device. There are two basic embodiments of the present invention that will be described in more detail below. For example, it is possible to use markers that are also visible in X-ray imaging and to record a localization image data record with an image recording device of the irradiation device as is known in principle. This makes it possible, once again in particular using segmentation algorithms, to determine the position of the markers in the localization image data record and therefore in the irradiation device so that the marker information is then available. However, in another variant that enables radiation planning, which is purely based on magnetic resonance imaging, the patient positioning device can only be coupled in one way, that is in a defined position and orientation, to the irradiation device so that, the marker information is known, since only one single possibility exists for this.

In this context, it is noted that it is also conceivable to dispense with the markers entirely if the magnetic resonance device is provided with a coupling apparatus that ensures a pre-specified positioning of the patient positioning plate in the magnetic resonance device. If a coupling apparatus of this kind is present both on the magnetic resonance device and on the irradiation device, location information on the target region relative to the patient positioning device that can be determined in the magnetic resonance image data record can be used directly to enable the irradiation device in which the position of the patient positioning device is also defined to identify the target region to be irradiated. However, since this is more complicated to implement on a magnetic resonance device, an embodiment of this kind is less preferable.

With an extension to a method for setting an irradiation device before irradiation of a patient, the target information determined with the method according to the invention can also be used for the automatic setting of the irradiation device. The target information, i.e. the position of the target region in the irradiation device, is used for the specific control of the irradiation device.

Overall, therefore, the present invention also enables use to be made of the repositioning precision of the irradiation device even without a computed-tomography-planning image data record and without error-prone bone depiction by the magnetic resonance imaging. Hence, with regular repetition of the magnetic resonance measurement before each individual irradiation process, the precision of the positioning can also be increased with displaceable target regions so that damage to sensitive organs, for example the bowel, can be reduced since lower error tolerances could be selected around the so-called “organs at risk” or the target region. Since a magnetic-resonance-compatible patient positioning device provided with markers is used on which the patient remains for the whole time, the suggested image-based method also eliminates a series of precision requirements for the actual magnetic resonance device, for example the travel precision of the patient couch of the magnetic resonance device and/or, if provided, the precision of the calibration of a magnetic resonance laser/simulation laser in the scanner chamber of the magnetic resonance device.

As mentioned above, in a first variant of the method according to the invention the marker information is determined by the evaluation of at least one localization image data record recorded by an image recording device of the irradiation device showing the markers also visible in X-ray imaging. Alternatively or also to achieve higher precision, it can additionally be provided in another variant of the invention that the marker information is determined by predetermination using a coupling apparatus that ensures a prespecified positioning of the patient positioning device in the irradiation device. In cases in which only one coupling apparatus is used in order to permit only one single position of the patient positioning device in the irradiation device, the position of the patient positioning device, and hence also of the markers in the irradiation device, is in principle known so that X-ray imaging can be dispensed with entirely and hence the stress on the patient caused by this can be avoided. Nevertheless, it can also be expedient, by low-dose imaging, optionally additionally to record localization imaging data records, wherein, in particular with a suitable choice of markers, here the doses should be selected to be extremely low and with as little stress as possible for the patient.

At least one two-dimensional or one three-dimensional X-ray image can be prepared as a localization image data record. Therefore, the portal images are just as suitable as three-dimensional X-ray images reconstructed from projection images taken under different projection directions, which can, for example, be determined by back projection. In this case, the recording arrangement of the image recording device comprising an X-ray tube and an X-ray detector facing each other can, for example, be swiveled jointly in the gantry around the patient. Three-dimensional localization imaging data records have the advantage that during evaluation, for example by segmentation, they can directly reflect the position of the markers in three dimensions. This is important since displacements of target regions can occur in all spatial directions and therefore three-dimensional target information fully exploiting the three-dimensional location information is required.

However, even with the use of at least one two-dimensional X-ray image as a localization image data record, there are possibilities for determining three-dimensional positions of the markers in the irradiation device. For example, it can be initially provided that the three-dimensional position of the markers in the irradiation device is determined from at least two two-dimensional X-ray images in particular recorded in projection directions perpendicular to each other. Here, it is possible to use known back projection techniques in order to determine a three-dimensional position of the markers. If only one single two-dimensional X-ray image is used as a localization image data record, the use of cone-beam geometry can be exploited. To be precise, in cone-beam geometry, the distance between two markers and the size in which the markers are depicted can give an indication of the distance of the markers from the X-ray tube, which, with a known arrangement and size of the markers in the support device, can also be quantified. However, the use of at least two X-ray images, which are in particular perpendicular to each other, or of a three-dimensional X-ray image is usually more accurate and so this procedure is preferred.

The markers can be automatically localized in the localization image data record by segmentation. Markers are generally embodied such that they are visualized as precisely as possible and very distinctly in the respective imaging variant, here X-ray imaging and magnetic resonance imaging. Accordingly, segmentation algorithms able to find markers of this kind are known. Here, it is noted at this point that the markers can also be selected favorably, for example in their shape, to facilitate their identification by automatic segmentation algorithms.

In an embodiment of the present invention, that the location information is determined as a displacement compared to a reference position of the target region to the markers determined from a reference image data record showing the target region and the markers recorded by magnetic resonance imaging. In many cases, it happens that, with multistage irradiation with a plurality of individual irradiation processes, which can, for example, follow each other with an interval of several days and/or several weeks, initially the irradiation is in principle planned with reference to a planning image data record, which is presently recorded by magnetic resonance imaging. From the planning time point to the first irradiation process, unless this takes place immediately, or between the individual irradiation processes, there will obviously be changes in the position of the target region within the patient's body. However, if at one time point only basic settings for the correct positioning of the patient are known, i.e. specifically the target region to be irradiated, in the irradiation device, it can be expedient, at later time points or during radiation processes, only to indicate the displacement from this reference position, i.e. the deviation therefrom, in order to enable a new correct positioning of the patient in the irradiation device. Therefore, with this embodiment of the method according to the invention, a reference image data record is considered, wherein the location information is determined as a displacement from this reference position of the target region to the markers. Ideally, when a coupling apparatus is used for defined positioning, target information on the reference position already exists so that the location information can be applied as a displacement directly to the reference target information in order to be able to position the target region correctly in the irradiation device.

In this case, a planning image data record used during planning of the irradiation therapy and/or a magnetic resonance image data record used for the preparation of a previous irradiation process can be used as the reference image data record. This procedure can be expediently used if a coupling apparatus to ensure a specific positioning of the patient positioning device in the irradiation device is provided from which then in particular a reference target position can be determined from the planning image data record via the (there absolute) location information. However, it is also expedient to use a magnetic resonance image data record to prepare a previous irradiation process, in particular the first irradiation process, because after this the new target information can in each case be determined by the application of location information describing a displacement. In the cases described here therefore, it is only necessary to determine the target information via the marker information and the location information once; after this, new derived information is obtained from the displacement of the new displacement relative to the reference location information.

Expediently, it is possible to use markers made of gold and/or another material visible in both magnetic resonance imaging and in X-ray imaging and/or comprising gold. Gold markers are particularly suitable since they are substantially inert and clearly visible in both magnetic resonance imaging and X-ray imaging, if necessary. Obviously, other types of markers are also conceivable depending upon whether they are required for both magnetic resonance imaging and X-ray imaging or whether markers for magnetic resonance imaging are sufficient.

In another embodiment of the invention, an automatic transportation device, in particular a shuttle system, is used to transport the patient. In this way, the patient can be transported from the magnetic resonance device to the irradiation device automatically and/or guided, for example using rails. Alternatively or additionally, the patient positioning device is at least partially guided via a rail system to the irradiation device. Together with the preferred arrangement of the irradiation device and the magnetic resonance device close to each other, embodiments of this kind increase the reliability of transporting the patient if possible without movement from the magnetic resonance device to the irradiation device.

In addition to the method, the invention also encompasses a position determination system for determining a position of a target region of a patient to be irradiated in an irradiation device. The position determination system has a patient positioning device with markers that are visible at least in magnetic resonance imaging, a magnetic resonance device for recording a magnetic resonance image data record showing the target area of the patient containing the target region to be irradiated together with the markers, and an evaluation device for determining location information describing the three-dimensional position of the target region relative to the markers from the magnetic resonance image data record. The evaluation device determines target information describing the position of the target region in the irradiation device using marker information indicating the position of the markers in the irradiation device and the location information. The position determination system is designed to implement the method according to the invention. All embodiments relating to the method according to the invention apply to the position determination system according to the invention.

The markers can be integrated in a patient positioning plate and/or a vacuum cushion of the patient positioning device. Therefore, there are in principle different possibilities for arranging the markers in a patient positioning device. Arrangement in a patient positioning plate, the shape of which is as invariable as possible, is preferred. However, it can also be expedient, for example, to arrange the markers for example in a vacuum cushion after this has been placed more closely on the patient; however, this embodiment is more expedient when no predetermination of the marker position using a coupling apparatus is to be performed.

### BRIEF DESCRIPTION OF THE DRAWINGS

FIG. 1 is a flowchart of the method according to the invention.

FIG. 2 shows a patient placed on a patient positioning device.

FIG. 3 schematically illustrates a magnetic resonance image data record in a first irradiation process.

FIG. 4 shows the position of a target region relative to a marker.

FIG. 5 schematically illustrates a magnetic resonance image data record in a second irradiation process.

FIG. 6 shows a position determination system according to the invention.

### DESCRIPTION OF THE PREFERRED EMBODIMENTS

The present invention is concerned with the positioning of a target region to be irradiated correctly in an irradiation device for irradiation therapy. To this end, target information is required that discloses the position of the target region in the irradiation device as precisely as possible. In the exemplary embodiment shown here by way of example, the target region under discussion is a patient's prostate; obviously, however, other soft-tissue target regions to be irradiated, for example, soft-tissue tumors lying outside the prostate and the like, are also conceivable.

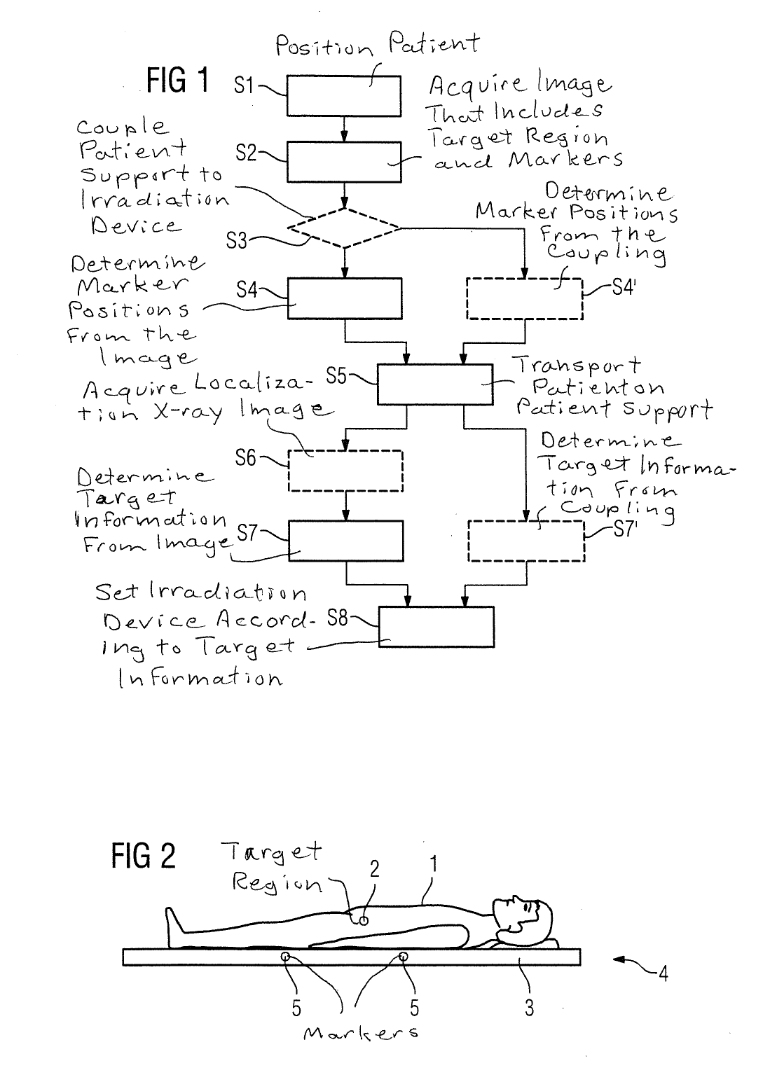

Various exemplary embodiments of the method according to the invention can be explained with reference to FIG. 1, wherein the flowchart shown relates to an irradiation process. In this case, irradiation therapy can be divided into several irradiation processes.

In a Step S1, the patient is positioned, and preferably also secured, on a patient positioning device. The patient positioning device is designed to be magnetic-resonance-compatible and comprises markers that are visible at least in magnetic resonance imaging.

This is shown as an example in FIG. 2, in which the patient 1 with the identified target region 2 is arranged on the patient positioning device 4 comprising a patient positioning plate 3. A number of markers 5, in the present case gold markers, for example, which are also visible in X-ray images, are integrated in the patient positioning plate 3. In alternative embodiments, markers 5 can also be integrated in a vacuum cushion or as a part of the patient positioning device 4.

Here, the patient positioning device 4 can embody multiple markers 5 in order to be able to cover target regions along the entire patient couch 3 as far as possible, wherein the markers, as is in principle known, can be designed with respect to their shape and/or size and/or other features such that they can be differentiated from one another. It is in principle also possible to use placeable markers 5, which are inserted in specially provided recesses of the patient positioning device 4 according to where they are required according to the location of the target region 2. In addition, the shape and/or size and/or other features of the markers 5 can be adjusted such that they are as easy as possible to find during the course of an automatic evaluation, in particular segmentation, of an image data record showing them and/or provide orientation information on the markers 5.

Still in Step S1 of FIG. 1, the patient 1 on the patient positioning device 4 is then introduced into a magnetic resonance device, wherein the patient positioning device 4 can, for example, be placed on a patient couch of the magnetic resonance device or replace the latter.

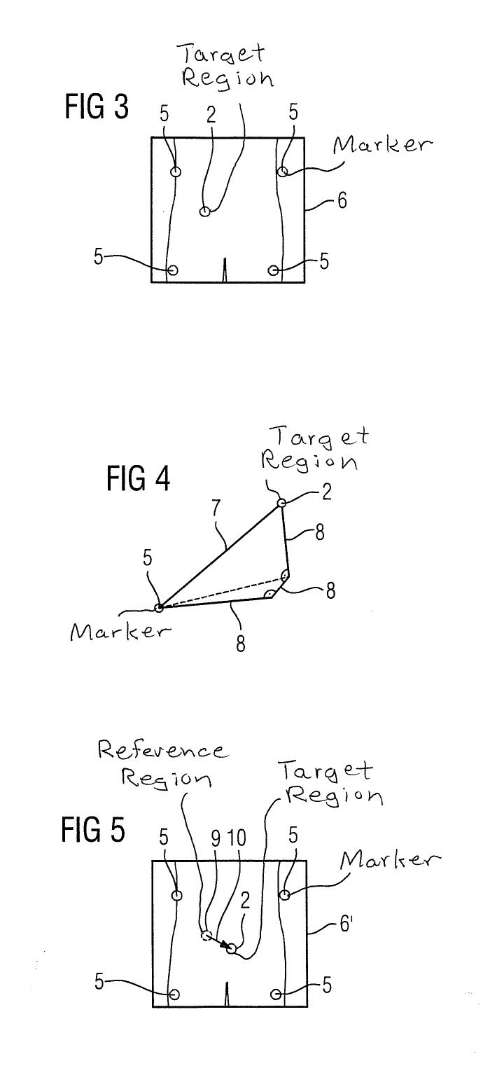

Then, in Step S2, a target area of the patient 1 containing the target region to be irradiated 2, i.e. in this case the prostate, is recorded such that the markers 5 can also be seen in the resulting magnetic resonance image data record. This is shown in a sketch in FIG. 3 as an abstracted magnetic resonance image data record 6 showing the markers 5 (or at least a part of the markers 5) and the target region 2. Therefore, the magnetic resonance image data record 6 offers a basis for the determination of the position of the target region 2 relative to the markers 5. To this end, FIG. 4 is an orientation sketch, wherein the target region 2 and a marker 5 are shown in their spatial relationship to each other, wherein their spatial position to each other is defined by a three-dimensional vector 7, which, as is in principle known, can be made up of three orthogonal components 8. Since there are a plurality of markers 5, finally, depending on whether the embodiment of the markers 5 is selected such that an absolute orientation of the vector 7 can already be determined therefrom, at the latest with the use of three markers 5, it is also possible to obtain information on the position of the target region 2 relative to the patient positioning device 4 in general.

Step S3 is optional and in the present case relates to an embodiment in which a coupling apparatus of the irradiation device ensures that the patient positioning device 4 can only be connected to the irradiation device in a specific position and orientation. Therefore, in this case, which will be described with in more detail below, the position of the markers 5 in the irradiation device is already predetermined and known.

Nevertheless, initially a variant of the method according to the invention is shown in which the position of the markers 5 in the irradiation device is not prespecified. This is now followed by a Step S4, which also has to be carried out at least once in the previously addressed variant of the method according to the invention, in which, in particular automatically, location information is determined which describes the position of the target region 2 relative to the markers 5, as indicated in FIG. 4. The target region 2 and the markers 5 can be identified in the magnetic resonance image data record 6 by segmentation, for which known segmentation algorithms can be used. Also conceivable are exemplary embodiments in which Step S4 is carried out at least partially manually.

In a Step S5, the patient positioning device 4 is then transported to the irradiation device without the patient 1 being moved. An automatic transportation device, for example a shuttle system comprising a rail system is used to this end. This ensures the guidance of the patient 1 on the patient positioning device 4 to the irradiation device with as little vibration as possible.

In the variant of the method according to the invention shown initially, the position of the markers 5 in the irradiation device is not known in advance so that in this case, in which markers 5 are also used which are also visible in X-ray imaging, a Step S6 is performed in which a localization image data record showing the markers 5 is recorded with an image recording device of the irradiation device. Then the three-dimensional position of the markers in the irradiation device is identified from this localization image data record, in particular by automatic evaluation, for example with segmentation algorithms. As already indicated, this position of the markers 5 in the irradiation device is referred to as marker information. Here, various variants are conceivable in Step S6.

It is preferable for the patient 1 to be exposed to the lowest possible X-ray dose and the use of markers 5 which are also visible with an extremely low dose makes this possible in an unproblematic way, wherein, however, it is also expedient to work with a few two-dimensional X-ray images as a localization image data record. Recording of two two-dimensional X-ray images standing perpendicular to each other makes it possible in a known manner by means of back projection to determine a three-dimensional position of the markers 5. Since, however, image recording devices of irradiation devices frequently also use cone-beam geometry, just one single X-ray image can be sufficient to localize the markers 5 three-dimensionally as well, since the display size of the markers 5 and the distance between the markers 5 depend on the radiation source of the image recording device.

However, it is also possible to use a three-dimensional localization image data record, which can be reconstructed from two-dimensional projection images with different projection directions as a three-dimensional X-ray image, for example by filtered back projection. To this end, the image recording device of the irradiation device, that is in particular the recording arrangement formed by the X-ray tube and X-ray detector, can expediently be rotated around the patient 1 in the gantry and the radiation source of the irradiation device. In a three-dimensional localization image data record, the position of the markers 5 with their segmentation is ultimately directly known.

If, alternatively, a coupling apparatus for the patient positioning device 4 is provided in the irradiation device, the marker information is predetermined and Step S6 can be omitted or is only used for checking purposes.

In a Step S7, since the position of the markers 5 in the irradiation device as marker information is known just as well as the position of the target region 2 relative to the markers 5 as location information, it is then possible to determine the target information indicating the position of the target region 2 in the irradiation device from the marker information and the location information.

In a Step S8, the target information can then be used in order automatically to set the irradiation device exactly to the target region 2.

As explained, a modified exemplary embodiment of the method according to the invention is conceivable and in particular advantageous when the marker information, i.e. the position of the markers 5 in the irradiation device, is known in advance due to the use of a coupling apparatus. Then, following the first pass of the method according to the invention, for example on the first irradiation process of a plurality of irradiation processes, the target information determined can be stored as reference target information. Now, if only the displacement of the target region 2 compared to the reference position of the target region 2 relative to the markers 5 assigned to the reference target information is determined as location information, it is easy to determine the current target position from the reference target information since only the displacement also has to be applied to the reference target position. Therefore, in this variant, a check is performed in the optional Step S3 as to whether reference target information is already available. If this is the case, in a Step S4′, the magnetic resonance image data record which was recorded in Step S2 is evaluated again, but in this case with respect to a possible displacement of the target region 2, which is explained in more detail in FIG. 5 by the magnetic resonance image data record 6′ depicted as abstracted. The markers 5 and the target region 2 are again clearly identifiable, wherein, however, the latter is clearly displaced compared to the reference position 9. The corresponding displacement 10 is now determined in Step S4′, once again by the automatic evaluation of the magnetic resonance image data record 6′.

Accordingly, following the transportation of the patient 1 to the irradiation device in Step S5, in a Step S7′ of this variant of the method according to the invention, the new target information is determined by applying the displacement to the reference target information before, in Step S8, the setting of the irradiation device is again performed with reference to the target information.

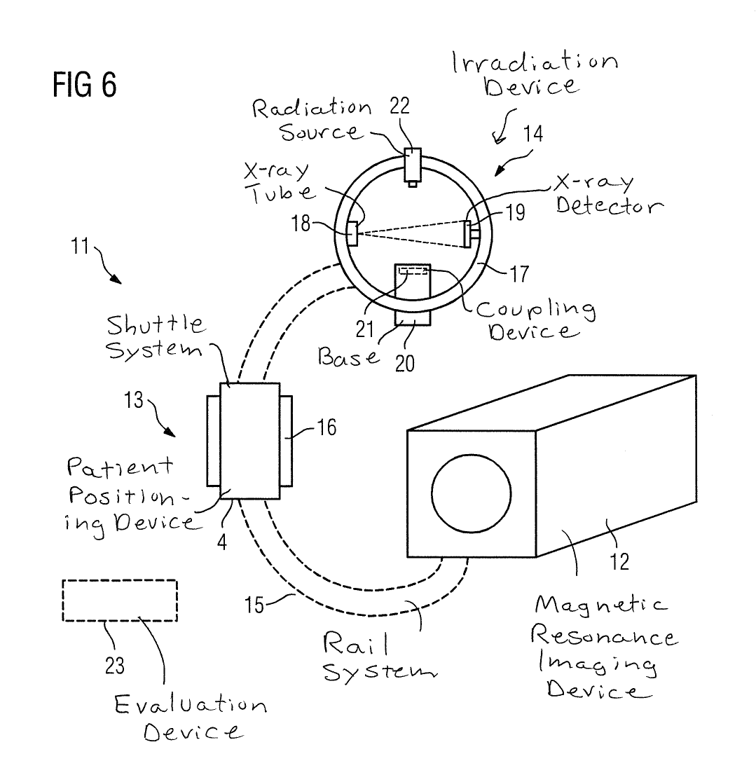

FIG. 6 shows a position determination system 11 according to the invention, in which the method according to the invention can be performed. As already discussed, this includes a magnetic resonance imaging device 12 as is in principle known in the prior art. The magnetic resonance image data record 6, 6′ can be recorded therewith. A transportation device 13 enables the patient positioning device 4 to be moved from the magnetic resonance device 12 at least partially automatically to the irradiation device 14. In the present case, the transportation device 13 includes a rail system 15 and a shuttle system 16 for the automation.

The irradiation device 14 has a gantry 17 along which the radiation source 22, for example a linear accelerator (LINAC) can be rotated about the patient 1 (who, due to the method according to the invention, is correctly positioned). Also arranged on the gantry is the recording arrangement of an image recording device of the irradiation device 14, formed by an X-ray tube 18 and an X-ray detector 19, which lie opposite each other and are able to record X-ray images in cone-beam geometry. The irradiation device 14 further comprises a base 20 on which the patient positioning device 4 can be mounted. Here, optionally, a coupling apparatus 21 is provided which can ensure the defined position and orientation of the patient positioning device 4 in the irradiation device 14.

The evaluation of magnetic resonance image data records 6, 6′ or, if provided, localization image data records, for example by means of segmentation algorithms, is automated by an evaluation device (computer) 23, which is only indicated here, and which is preferably made up of control devices of the magnetic resonance device 12 and the irradiation device 14.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for determining a position of a target region of a patient to be irradiated in an irradiation apparatus, said method comprising:
positioning a patient on a patient positioning device comprising markers that are visible at least in magnetic resonance images;
operating a magnetic resonance scanner to acquire a magnetic resonance image data record from the patient, showing a target area of the patient that contains the target region, together with said markers;
evaluating said magnetic resonance image data record to determine location information therefrom that describes a three-dimensional position of said target region relative to said markers;
transporting the patient on the patient positioning device, with the patient being unmoved from said patient position on said patient positioning device, to said irradiation device; and
determining target information describing the position of the target region in the irradiation device using information identifying a position of said markers in said irradiation device, and said location information.

2. A method as claimed in claim 1 comprising automatically, in a computer, evaluating said magnetic resonance image data record to determine said location information, and to determine said target information describing the position of the target region in the irradiation device.

3. A method as claimed in claim 1 wherein said markers are also visible in x-ray images, and comprising acquiring a localization image data record, showing the target area with the markers, from the patient with an x-ray imaging device, and determining said marker information by evaluating said localization image data record together with said magnetic resonance image data record.

4. A method as claimed in claim 3 comprising acquiring said localization image data record as a two-dimensional x-ray image or as a three-dimensional x-ray image.

5. A method as claimed in claim 3 comprising acquiring said localization image data record to comprise at least two two-dimensional x-ray images, and determining the three-dimensional position of the markers from said at least two two-dimensional x-ray images.

6. A method as claimed in claim 5 comprising acquiring said two two-dimensional x-ray images as projection images respectively in projection directions that are perpendicularly to each other.

7. A method as claimed in claim 3 comprising acquiring said localization image data record by operating said x-ray imaging device with a cone-beam geometry.

8. A method as claimed in claim 1 comprising determining said three-dimensional position of said target region in a computer, by executing a segmentation algorithm in said computer within which said markers are segmented.

9. A method as claimed in claim 1 comprising determining said marker information from a coupling apparatus that ensures a predetermined positioning of the patient positioning device in said irradiation device.

10. A method as claimed in claim 1 comprising determining said location information as a displacement of said markers compared to a reference position of the target region relative to said markers, determined from a reference image data record showing the target region and the markers acquired by magnetic resonance imaging.

11. A method as claimed in claim 10 comprising using, as said reference image data record, an image data record selected from the group consisting of a planning image data record used for planning irradiation therapy to be implemented in said irradiation device, and a previous magnetic resonance image data record used for preparing previous irradiation in said irradiation device.

12. A method as claimed in claim 1 comprising forming said markers at least partially from gold.

13. A method as claimed in claim 1 comprising transmitting said patient with an automatic patient transportation device.

14. A method as claimed in claim 13 comprising transporting said patient with an automated shovel system.

15. A method as claimed in claim 1 comprising transporting said patient positioning device to said irradiation device on a rail system.

16. A patient position determination system comprising:
a patient positioning device comprising markers that are visible at least in magnetic resonance images;
a magnetic resonance scanner;
a control computer configured to operate said magnetic resonance scanner to acquire a magnetic resonance image data record from a patient said patient positioning device, showing a target area of the patient that contains the target region, together with said markers;
said control computer being configured to evaluate said magnetic resonance image data record to determine location information therefrom that describes a three-dimensional position of said target region relative to said markers;
said patient positioning deice being configured to transport the patient on the patient positioning device, with the patient being unmoved from said patient position on said patient positioning device, to said irradiation device; and
a computer at said irradiation device configured to determine target information describing the position of the target region in the irradiation device using information identifying a position of said markers in said irradiation device, and said location information and to operate the irradiation device to irradiate the target region.

17. A patient position determination system as claimed in claim 15 wherein said markers are integrated in said patient positioning device.

18. A patient position determination system as claimed 15 wherein said patient positioning device comprises a vacuum cushion on which said patient is situated, and wherein said markers are integrated in said vacuum cushion.
